(19) United States
(12) Patent Application Publication
(10) Pub. No.: US 2025/0182284 A1
(43) Pub. Date: Jun. 5, 2025

(54) IMAGE PROCESSING FOR DETERMINING A PHYSIOLOGICAL PARAMETER

(71) Applicant: I-VIRTUAL, Metz (FR)

(72) Inventors: Abdelhak MOUSSAOUI, Vigny (FR); Alain PRUSKI, Scy-Chazelles (FR)

(21) Appl. No.: 18/854,025

(22) PCT Filed: Apr. 3, 2023
(86) PCT No.: PCT/EP2023/058618

§ 371 Date: Oct. 3, 2024

(30) Foreign Application Priority Data
Apr. 7, 2022 (FR) 2203190

(51) Int. Cl. G06T 7/00; G06V 10/75; G16H 30/40

(52) U.S. Cl. CPC G06T 7/0016; G06V 10/751; G16H 30/40

(57) ABSTRACT

A method for determining a physiological parameter such as a heart rate of an individual based on a source image stream (IS1, IS2, . . . ISM), including generating averaged images and an operation of individually selecting pixels in each of the averaged images on the basis of a variation over time in the value of these pixels is disclosed.

### TECHNICAL FIELD

The invention relates to the field of image processing.

The invention is of particular interest in the field of non-contact photoplethysmography.

### PRIOR ART

Non-contact photoplethysmography can be used to determine a physiological parameter of an individual such as their heart rate, that is the average number of heart muscle contractions in one minute, known as “beats per minute”.

Generally speaking, this non-invasive optical technique involves assessing variations in blood volume in superficial tissue on the basis of variations in light absorption in these tissues.

Known non-contact photoplethysmography techniques provide measurements whose accuracy and reliability are highly dependent on variations in ambient lighting and individual movement, leading to measurement errors.

### DISCLOSURE OF THE INVENTION

One aim of the invention is to reduce such measurement errors.

Another aim of the invention is to provide an image processing solution compatible with the acquisition of these images using different types of camera, for example monochrome or polychrome cameras, and/or under different lighting conditions, for example in the absence of visible light.

To this end, the object of the invention is a method for determining a physiological parameter of an individual from a stream, called “source stream”, of images of a region of the individual's body, this method comprising the following steps:
generating a stream of images, called an “averaged stream”, each obtained by averaging a plurality of successive images of the source stream selected by moving a sliding window;
generating a stream, called the “processed stream”, of images each obtained from two respective successive images of the averaged stream so that, for each of the images of the processed stream, one or more pixels of this image has a value:
equal to the value of a corresponding pixel of one of said two respective images of the averaged stream when the absolute value of the difference between this value and the value of a corresponding pixel of the other of said two respective images of the averaged stream is greater than a first threshold and less than a second threshold,
equal to zero otherwise;
determining said physiological parameter from variations in the value of pixels in images of the processed stream.

Conventionally, an image comprises a set of pixels forming a matrix of numbers, each representing a luminous intensity of a respective one of these pixels. The value of a pixel is its luminous intensity. For the purposes of the present invention, an image is therefore a series of numbers that do not necessarily take the form of a visual representation.

From a terminological point of view, a “corresponding” pixel in one image, relative to a pixel in another image, is a pixel with the same coordinate. For example, in a matrix coordinate system, a coordinate pixel (2, 3) of a first image is a pixel corresponding to the coordinate pixel (2, 3) of a second image, and vice versa.

In particular, the step of generating the averaged stream makes it possible to reduce the noise present in the source stream, by averaging images from that source stream. More precisely, for each series of images in the source stream undergoing this operation, averaging is preferably performed by calculating the average of the respective values of corresponding pixels in the images in this series, individually for each pixel. In other words, the value of each pixel in each of the averaged stream images preferably corresponds to the average of the values of corresponding pixels from images in the source stream image series used to generate that averaged stream image.

The method of the invention as defined above, and in particular the step of generating the processed stream, enables a physiological parameter to be determined on the basis of an individual selection of pixels from images of the averaged stream, disregarding pixels with light intensity variations that are too small or too large from one image of the averaged stream to the next. In particular, this makes it possible to reject any data that is not representative of an actual change over time in the physiological parameter of interest, but rather, for example, of a change in brightness or ambient lighting.

The result is a reduction in errors in determining the physiological parameter, while providing a corresponding confidence index, the latter being proportional to the number of non-zero pixels in the processed stream images.

In addition, such image processing reduces constraints on the luminous properties of the signals represented by the images of the source stream. In particular, a simple variation in contrast is enough to vary the luminous intensity of one or more pixels from one image to another. In this way, the method is fully functional when the source stream is acquired in the presence of backlighting or a lack of light, or when the source stream represents electromagnetic waves in the near-infrared range.

More generally, the invention enables reliable and accurate results to be obtained from a stream of images that can be acquired under a variety of lighting conditions and using different types of camera, such as a monochrome or trichrome camera.

The physiological parameter is preferably a vital biological parameter such as heart rate. Other physiological parameters can be determined using the method of the invention, for example heart rate variability, blood pressure or respiratory rate. These examples are non-limiting.

In one embodiment, said sliding window is moved with a step smaller than the number of images in the source stream used to obtain any one of the averaged stream images.

In other words, the step with which the sliding window is moved is preferably smaller than the size of that window.

By way of example, this step can be one or two images.

In one embodiment, the number of source stream images used to obtain each of the averaged stream images is odd, for example 3 or 5.

In one embodiment, said first threshold is equal to 0.

In one embodiment, said second threshold is equal to 1/((N1−1)*K), where N1 corresponds to the number of values that the pixels of the source stream images can have, and K corresponds to the number of source stream images used to obtain each of the averaged stream images.

In one embodiment, the method comprises generating a signal from values that each correspond to the average value of pixels in a respective one of the images in the processed stream whose value is non-zero.

In other words, for each of the images in the processed stream, the non-zero pixels in that image can be averaged to form a respective one of the values of that signal.

Preferably, the physiological parameter is determined on the basis of that signal.

In one embodiment, the method comprises a step of acquiring the source stream using a camera.

In a first embodiment, the camera is monochrome.

In a second embodiment, the camera is polychrome.

In this second variant, it is preferred that the values of the pixels correspond to the luminous intensity of a single color channel.

This simplifies processing.

For example, this color could be green.

Of course, a plurality of color channels can be subjected to the processing described above simultaneously.

In one embodiment, the method further comprises, prior to acquisition of the source stream, a step of setting the exposure and/or brightness parameters of the camera and, during acquisition of the source stream, maintaining the values of these parameters.

Such a setting optimizes the useful information present in the source stream images and reduces information due to measurement artifacts.

The invention also relates to a device comprising means adapted to perform the steps of the method as defined above.

In one embodiment, this device further comprises a camera configured to perform the source stream acquisition step of the method as defined above.

By way of example, this camera may belong to a digital device such as a cell phone, tablet or computer.

Furthermore, the invention relates to a computer program comprising executable computing instructions which, when executed by computer, implement the steps of the method as defined above.

Further advantages and features of the invention will become apparent from the following detailed, non-limiting description.

### BRIEF DESCRIPTION OF THE DRAWINGS

The following detailed description makes reference to the accompanying drawings wherein:

FIG. 1 is a diagram showing the steps of a method according to the invention;

FIG. 2 is a schematic view of a source image stream;

FIG. 3 is a schematic view showing the generating of a first image of an averaged stream from a first series of images of the source stream;

FIG. 4 is a schematic view showing the generating of a second image of an averaged stream from a second series of images of the source stream;

FIG. 5 is a schematic view of a processed image stream, generated from the averaged stream.

### DETAILED DESCRIPTION OF EMBODIMENTS

The following is an example of a method in accordance with the invention, in particular for determining an individual's heart rate. This method or variants of this method can be implemented in a similar way to detect another physiological parameter such as heart rate variability, blood pressure, breathing rate, or even the oxygen saturation level of this individual.

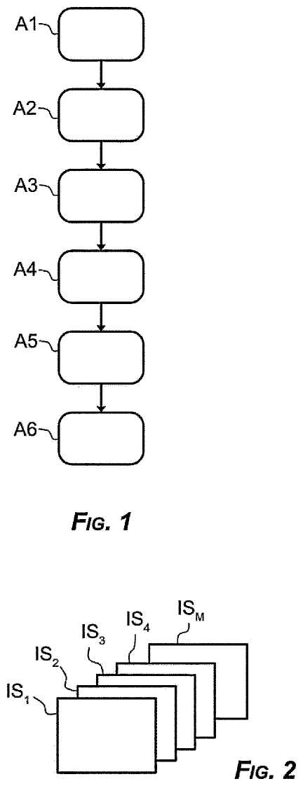

In this non-limiting example, the method successively comprises the following steps, which are shown in the diagram of [FIG. 1]:
A1: setting up a digital camera;
A2: acquiring an image stream, called the “source stream”;
A3: generating a stream of images, called an “averaged stream”, from the source stream;
A4: generating a stream of images, called the “processed stream”, from the averaged stream;
A5: determining the physiological parameter;
A6: displaying the result.

In this example, the camera is monochrome and acquires digital images each comprising a number of B1*B2 pixels.

In a manner known per se, for each pixel P(C1,C2) of each image organized in matrix form, C1 is a first pixel coordinate corresponding, for example, to a column number of the matrix and C2 is a second pixel coordinate corresponding, in this example, to a row number of the matrix. B1 thus corresponds to the number of columns and B2 to the number of rows in the matrix, so that each pixel has a C1 coordinate between 1 and B1 and a C2 coordinate between 1 and B2.

In this example, B1 is 4000 and B2 is 3000, so that each image acquired with this camera has a resolution of 12 million pixels.

The value I(C1,C2) of each pixel P(C1,C2) represents in this example its luminous intensity in grayscale and is a natural integer between 0 and 255. Each pixel P(C1,C2) can thus have 256 values.

Step A1 comprises setting the camera brightness to a given value and adjusting the camera exposure, using a computer program designed to modify the exposure incrementally from zero exposure until the mean value I_{moy}(C1,C2) of each pixel P(C1,C2) of a series of successively acquired images lies between a lower threshold and an upper threshold, for example between 160 and 200. In this example, the series of images acquired for this purpose comprises 30 frames per second.

If this procedure fails to produce an average value I_{moy}(C1,C2) for each pixel P(C1,C2) between these upper and lower thresholds, the program modifies the brightness and then repeats the exposure adjustment as described above, and so on until the above conditions are met.

The exposure and brightness values thus obtained are maintained during the A2 acquisition step, thus avoiding the measurement artifacts that would result from automatic adjustment of these parameters.

The camera is then arranged to acquire images of a region of the individual's body, in this example part of the face.

[FIG. 2] shows schematically a succession of M images ISᵢ (i ranging from 1 to M) acquired in this way and constituting said source stream. These images ISᵢ are called “source images”.

The source stream thus acquired is representative of a temporal variation in light information backscattered by superficial tissue of the individual's face, corresponding to a temporal variation in blood volume in vessels running through that tissue, a greater blood volume resulting in greater light absorption by the oxygen contained in the blood.

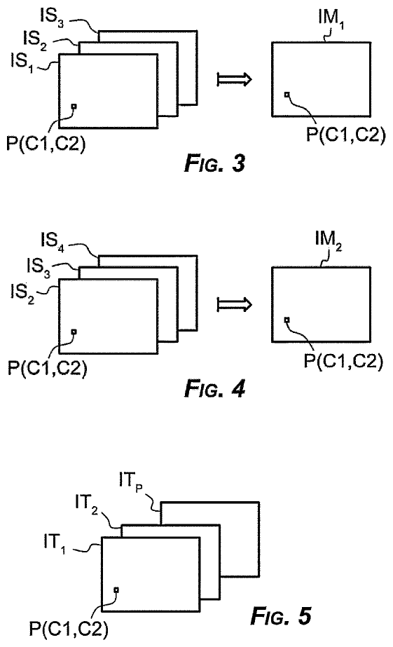

Step A3 for generating said averaged stream is described below with reference to FIGS. 3 and 4.

To generate the averaged stream, a plurality of series are created, each containing K images of the source stream, K being generally greater than 1 and less than M, and in this example equal to 3.

The first series, shown in [FIG. 3], comprises the first three images IS₁, IS₂ and IS₃ of the source stream.

The second series, shown in [FIG. 4], comprises images IS₂, IS₃ and IS₄ of the source stream, that is the three consecutive images starting from the second image IS₂ of the source stream.

The third series, not shown, comprises three consecutive images of the source stream starting from the third image IS₃.

More generally, each of the series comprises K consecutive images of the source stream, starting from a respective image of the source stream, thus forming a number of L=M−(K−1) series.

In other words, these L series of K successive source images are selected by moving a sliding window with, in this example, a step of 1 image.

For each of the series thus constituted, the source images of that series are averaged so as to form a respective one of the images IMⱼ (j ranging from 1 to L) of said averaged stream.

Thus, with reference to [FIG. 3], the value of each of the pixels P(C1,C2) of the averaged image IM₁ resulting from the averaging of the source images IS₁, IS₂ and IS₃ of the first series is equal to the average of the values of this pixel P(C1,C2) on each of the source images IS₁, IS₂ and IS₃.

The same applies to producing the averaged image IM₂ resulting from the averaging of the source images IS₂, IS₃ and IS₄ of the second series ([FIG. 4]), and for the other images of the averaged stream.

Given these averaging calculations, the value of each pixel P(C1,C2) in the averaged images IMⱼ is a real number, compared to the value of each pixel in the source images, which is a natural integer.

Step A4 for generating the processed stream is now described.

The processed stream comprises a number P of images ITₙ, referred to as “processed images”, each comprising a portion of the information from the averaged images IMⱼ, with j ranging from 1 to L−1, respectively.

In this example, the processed stream comprises one image less than the averaged stream.

More precisely, with regard to the first image IT₁ of the processed stream, each of the pixels P(C1,C2) of this image IT₁ has a value which is equal to the value of the corresponding pixel P(C1,C2) of the averaged image IM₁ if the absolute value of the difference between this value and the value of the corresponding pixel P(C1,C2) of the next averaged image, that is in this case the image IM₂, is greater than a first threshold S1 and less than a second threshold S2. If this absolute value is less than or equal to 1, or greater than or equal to S2, the corresponding pixel P(C1,C2) of this image IT₁ has a value equal to zero.

In other words, the image IT₁ of the processed stream retains the pixels of the image IM₁ of the averaged stream when the variation in their value between the image IM₁ and the image IM₂ lies between thresholds S1 and S2, and replaces the value of the other pixels with a zero value.

The second image IT₂ of the processed stream is obtained in a similar way from the averaged images IM₂ and IM₃.

The same applies to images IT₃ to IT_{P}, which are obtained from images IM₃ to IM_{L}, that is by moving a sliding window that selects two successive averaged images at each iteration, this window being moved by a 1image step in this example.

In this particular example, the threshold S1 is equal to 0 and the threshold S2 is equal to 1/((N1−1)*K), N1 being the number of values that the pixels of the source stream images can have, that is in this example 256. These thresholds can be determined empirically, depending, for example, on the nature of the physiological parameter to be determined.

From a temporal point of view, each of the images IMⱼ of the averaged stream is positioned at the same level as the intermediate image of the source image series used to generate this averaged image. For example, the first image IM₁ of the averaged stream corresponds to the temporal position of the source image IS₂. As a result, in this example, the first image IT₁ of the processed stream is moved in time by one frame relative to the first image IS₁ of the source stream.

Taking as an example an acquisition of 30 source images per second, this results in a time lag of 1/30 seconds between the processed stream and the source stream.

The physiological parameter, in this example the heart rate, can be determined A5 on the basis of the processed stream thus obtained.

In this non-limiting example, a time signal is formed, each point of which has an amplitude equal to the average value of the non-zero pixels of a respective one of the images in the processed stream. Thus, the first point of this signal has an amplitude corresponding to the average value of the non-zero pixels present in the first image IT₁ of the processed stream, and so on for the following points.

Additional filtering is optionally performed if the amplitudes of two successive points of the signal differ too much from one another, for example by more than 20%. By way of example, the amplitude of the second point can then be reduced so that its deviation corresponds to a variation of 20%.

In this example, in step A6, the result is displayed on a screen. This display may comprise a visual representation of said signal, with and/or without additional filtering, and/or one or more values representative of heart rate and/or another physiological parameter calculated on the basis of this signal according to techniques which are well known as such.

The various steps just described are implemented using a computer program comprising corresponding executable computer instructions.

The above description is not limiting, and many variants based on it can be considered without going beyond the scope of the invention. For example, the camera may be of a different type, such as a multi-color cellphone camera. In this case, the above description can be applied analogously for each of the color channels, on the understanding that processing can be carried out on the basis of the pixel value of a single color channel, for example green. Alternatively, the processing described above can be carried out on the basis of values combining information provided by a plurality of color channels.

The numbers given in this description are examples only. In particular, the images can contain a number of pixels greater or less than 12 million. K can have a value other than 3, for example 5 or another preferably odd number. The sliding window step can be greater than 1 frame, for both generating the averaged stream and/or generating the processed stream.

For another example, said temporal signal on the basis of which the physiological parameter is determined can be constructed not by calculating the average value of the pixels of each of the processed images but by using the values of one or more given pixels having, for example, a non-zero value over a plurality of successive processed images.

1. A method for determining a physiological parameter such as a heart rate of an individual from a stream, called the “source stream”, of images of a region of the individual's body, said method comprising the following steps:
generating a stream of images, called the “averaged stream”, each obtained by averaging a plurality of successive images of the source stream selected by moving a sliding window; and
generating a stream, called the “processed stream”, of images each obtained from two respective successive images of the averaged stream so that, for each of the images of the processed stream, one or more pixels of this image has a value:
equal to the value of a corresponding pixel of one of said two respective images of the averaged stream when the absolute value of the difference between this value and the value of a corresponding pixel of the other of said two respective images of the averaged stream is greater than a first threshold and less than a second threshold,
equal to zero otherwise;
determining said physiological parameter from variations in the value of pixels in images of the processed stream.

2. The method according to claim 1, wherein said sliding window is displaced by a step smaller than the number of frames of the source stream used to obtain any one of the frames of the averaged stream, this step being, for example, one frame.

3. The method according to claim 1, wherein said first threshold is equal to 0 and said second threshold is equal to 1/((N1−1)*K), where N1 corresponds to the number of values that the pixels of the images of the source stream can have, K corresponding to the number of source stream images used to obtain each of the averaged stream images.

4. The method according to claim 1, comprising generation of a signal from values which each correspond to the mean value of the pixels of a respective one of the images of the processed stream whose value is different from zero, the determination of the physiological parameter being carried out on the basis of that signal.

5. The method according to claim 1, comprising a step of acquiring the source stream using a camera.

6. The method according to claim 5, wherein the camera is monochrome or polychrome, the pixel value preferably corresponding to the light intensity of a single color channel when the camera is polychrome.

7. The method according to claim 5, further comprising, prior to acquisition of the source stream, a step of setting the exposure and/or brightness parameters of the camera and, during acquisition of the source stream, maintaining the values of these parameters.

8. A device comprising means adapted to perform the steps of the method according to claim 1.

9. The device according to claim 8, further comprising a camera, belonging for example to a digital device such as a cell phone, tablet or computer, the camera being configured to perform an acquisition step of a source stream of a method for determining a physiological parameter such as a heart rate of an individual from a stream, called the “source stream”, of images of a region of the individual's body, said method comprising the following steps:
generating a stream of images, called the “averaged stream”, each obtained by averaging a plurality of successive images of the source stream selected by moving a sliding window, and
generating a stream, called the “processed stream”, of images each obtained from two respective successive images of the averaged stream so that, for each of the images of the processed stream, one or more pixels of this image has a value:
equal to the value of a corresponding pixel of one of said two respective images of the averaged stream when the absolute value of the difference between this value and the value of a corresponding pixel of the other of said two respective images of the averaged stream is greater than a first threshold and less than a second threshold,
equal to zero otherwise,
determining said physiological parameter from variations in the value of pixels in images of the processed stream, and
comprising a step of acquiring the source stream using a camera.

10. A computer program comprising executable computing instructions which, when they are executed by a computer, implement the steps of the method as claimed in claim 1.
